(19) United States
(12) Patent Application Publication
(10) Pub. No.: US 2006/0153906 A1
(43) Pub. Date: Jul. 13, 2006

(54) Pharmaceutical product for endonasal administration for treating central nervous system disease and disorders

(76) Inventors: Naum Goldstein, Berlin (DE); Roman Goldstein, Berlin (DE)

Correspondence Address: NORRIS, MCLAUGHLIN & MARCUS, 875 THIRD AVE, 18TH FLOOR, NEW YORK, NY, 10022, US

(21) Appl. No.: 10/527,941

(22) PCT Filed: Oct. 11, 2003
(86) PCT No.: PCT/DE03/03376

§ 371 Date: Sep. 23, 2005

(30) Foreign Application Priority Data
Oct. 17, 2002 (DE) 102 48 601.8

(51) Int. Cl. A61K 31/7076; A61K 38/12; A61K 31/70; A61K 31/515; A61K 31/485; A61K 31/519

(52) U.S. Cl. 424/450; 424/718; 514/9; 514/23; 514/45; 514/419; 514/270; 514/282; 514/561; 514/649; 977/907; 514/252.16; 514/253.04

(57) ABSTRACT

The invention concerns a pharmaceutical product consisting of one or more standard active substances and/or of one or more standard metabolites acting on the central nervous system. Said active substances are added with substances acting on the nasal mucous membrane for endonasal administration.

The invention relates to a pharmaceutical product with active agents affecting the central nervous system, with an added active substance acting on the nasal mucous membrane for endonasal administration.

The therapeutic use of oxygen radicals and/or their reaction or breakdown products is known. For example, the use of O₂^{•-} for the treatment of bronchial asthma is known (N. Goldstein et al., Adjuvante Inhalationstherapie des Asthma bronchial emit exogenem Superoxid (Adjuvant inhalation therapy of bronchial asthma with exogenic peroxide); Phys. Rehab. Kur. Med. 7,1997,138-140). It is also known that inhalation of gas phase peroxide or hydrogen peroxide in low concentrations enhances the analgesic effect of analgesics administered enterally or parenterally or intraperoneally, as described by Goldstein et al. (see Goldstein et al.: Exogenous gaseous superoxide potentiates the antinociceptive effect of opioid analgesic agents, Inflamm. Res., 45, 1996, 473-478), and known from DE 195 14 522. It is also known from DE 197 08 643 to use gaseous superoxide or hydrogen peroxide for a therapeutic administration with Morbus Parkinson.

One of the main possible methods for treatment and regulation of the functions of the organism of mammals is a direct stimulation or effect on the limbic and reticular system of the brain by affecting the hypothalamus. The hypothalamus is an important part of the brain, responsible for controlling the internal environment and maintaining the homeostasis of the organism.

Integrative functions of the hypothalamus include vegetative, somatic and hormonal reactions of the organism, in particular control of the activity of endocrinal and exocrinal glands, control of the sleep/wake state, sensation of pain, sensation of thirst and hunger, hormonal control, emotional reactions and the like. The particularities of the structure and function of the hypothalamus make it the target for adequate and non-invasive, non-traumatic treatment methods and for affecting different defective functions of the organism.

Special neurons of the hypothalamus have chemo-receptor functions and are sensitive to changes of physiologically important parameters in the blood and the cerebrospinal fluid. They are therefore capable of receiving information signals from the interior of the organism and from the environment. The information signals from the interior environment of the organism are transmitted through simple metabolites, for example by amino acids, carbohydrates, peptides, nucleotides. Hormones and their derivates, mediators and/or neuro-transmitters and other natural and artificial regulators also participate in the transmission of these signals.

The information signals from the environment, for example from inhaled air, are sensed by the hypothalamus through the exterior receptors of the nasal cavity and cause corresponding reactions of the organism. The hypothalamus is therefore a suitable point of attack for drugs and metabolic molecules for controlling physiological effects.

Unlike enteral or parenteral methods for the administration of drugs, the endonasal administration can be advantageous due to the significantly smaller concentrations and doses compared to the commonly used concentrations and doses. These advantages include primarily a reduction of toxic effects and of side effects. Another important advantage of the nasal administration of drugs is the non-invasive character.

However, DE 195 14 522 C1 describes potentiation of analgesics only in the context of SAR (oxygen ion radicals), relating exclusively to a separate administration of SAR (i/nasal) and analgesics (i/p and per os, respectively); moreover, the analgesic was not applied intranasally. The practical administration of these products is complicated, because they are based on two consecutive administrations, namely SAR and analgesics, and only moderately enhance the analgesic effect, whereas the therapeutic intranasal administration of SAR in treatment of Morbus Parkinson is surprisingly effective.

DE 693 21 458 T2 describes the administration of products that promote absorption. However, this relates to a form of an enhanced administration, but not to potentiation of the efficacy of particular types of analgesics. Only polar metabolites, such as glucoronides, of opioid analgesics were used for the therapeutic administration of opioid analgesics, which are supposed to have a greater efficacy compared to the base materials (morphine, codeine, levorphanol, etc.). The analgesic effect should develop at a concentration of morphine-6-glucoronide of 0.15 mg/kg bodyweight.

Only polar metabolites of opioid analgesics are described for therapeutic use which, however, are not suitable per se for generating an analgesic effect with morphine. Moreover, the drug form of an analgesic should only be prepared in connection with absorption-enhancing substances made of a cationic polymer, a bio-adhesive material, a surface-active material, a fatty acid, a helenate former, a mucus-dissolving substance, a cyclodextrin or a microsphere preparation.

Finally, DE 690 17 690 T2 describes administering, but not potentiating the efficacy of different drug preparations or metabolites. The compositions include absorption enhancers, namely surface-active substances, of a non-ionic type and of bile salt derivates and a drug, such as insulin or calcitonin.

Attempts of a direct endonasal administration of drugs are also described in the published literature. For example, the endonasal introduction of 6-glucoronide of morphine for reducing the side effects of the drug is known (Illum L. et al., 1996, Biopharm Drug Dispos., 17(8): 717-724).

Another example describes the endonasal administration of bromocriptin in an attempt to reduce the side effects on the gastrointestinal tract (Cicinelli E. et al., 1996, J. Endocrinol. Invest. 19(7): 427-432).

The hypothalamus is a suitable point of attack for endonasally applied drugs for affecting the central nervous system, as described for example by Illum L. et al. (1996), and Bechgaard E. et al. (J. Pharmacol., 1997, 49 (8): 747-750). In addition, RU-PS 2 149 043 describes a method for treating cerebral angiodystonia by endonasal administration of an aerosol containing lituitrin.

In a number of cases, natural hormones and their synthetic analogues and/or derivates are also used endonasally. For example, estrens are known which indirectly affect the hypothalamus. The administration of steroids is described in patent WO 9610032, authors: D. Berliner et al., for example 1,3,5 (10)-16-estratetraen-3-ol, which can stimulate the neuro-epithelial receptors of the nasal mucous membrane and can be introduced through the nasal cavity of the recipients. Also known is the endonasal administration of norpregnan for controlling sexual behavior.

A significant deficiency of the aforedescribed drugs for endonasal administration that can also affect the central nervous system is their unsatisfactory healing efficacy. The reason for the low efficacy of prior art endonasally applied drugs may be caused by the inadequate sensibility of the receptor structures of the target organs, in particular the cores of the hypothalamus and other structures of the brain, or in the high threshold for stimulating the affected receptors. Endonasally applied drugs that affect the central nervous system have therefore only rarely been used.

It is an object of the present invention to increase the efficacy of drugs, metabolites and/or other reactive agents of the aforedescribed type that can be used for treating disorders of the central nervous system, to significantly reduce the normally used dosage, and to achieve a faster, prolonged activity.

The object is solved by administering a combined composition of free-radical products with biologically active ingredients for potentiating the efficacy, whereby potentiation is achieved in combination with oxygen anion radicals (SAR) and/or nitrogen oxide-active products.

With these measures, subsequently abbreviated as NSAS, a pharmaceutical substance is produced, which can be used to initially sensitize the mucosa membrane of a patient. It is then possible to use different substances, including metabolites. The increased efficacy is not only produced in combination with SAR, but also with NO-active products. Only a combined composition of free-radical products with biologically active substances is administered. The administration becomes much simpler in practice, and a significantly higher analgesic effect is achieved with the same dosage, or the same effects are achieved with a significantly lower dosage of analgesics.

It is also possible to administer pharmaceutically effective substances, which are typically administered in form of cabinets, drops, injection and the like, as a nasal spray or a nasal ointment. The pharmaceutically effective substances can therefore be administered in a significantly smaller dosage, achieving nevertheless the same effect as with the current higher doses.

In one embodiment, the NSAS oxygen radicals and/or their reaction or decomposition products, namely perhydroxyl radicals, can be hydrogen peroxide, hydro-peroxide radicals or their hydrate clusters, and the substances active in the nasal mucus membrane can be forms of nitrogen mono-oxide (NO) and their precursors or reaction products.

Additional advantageous measures are described in the dependent claims. The invention will now be described in more detail.

It was unexpectedly discovered that by adding so-called vaso- or NSAS to drugs acting on the central nervous system, a significant increase in the efficacy of the respective drug can be achieved, if the drug and the NSAS are applied in form of a mixture.

Included in the term vasoactive substances or substances that act on the nasal mucosa membrane according to the invention are in particular the oxygen radicals or radical formers O₂^{•-}, H₂O₂, —O₂H, their hydrate clusters and also singlet oxygen ¹O₂, namely vasoactive forms of NO and biochemical substances, such as arginin, bradykinin, urea, which have a similar physiological effects in the context of the invention.

It is significant for the invention that the oxygen anion radicals are applied simultaneously with the drug in form of an endonasal applied mixture, preferably in liquid form.

Because of their metastable state, the oxygen anion radicals and/or nitrogen oxide are mixed with the respective drug immediately before administration of the pharmaceutical composition.

The oxygen anion radicals of the present invention can be formed by chemical or enzyme generation, for example by xanthine oxidase (Fridovich, I., (1970), “Quantitative aspects of the production of superoxide anion radical by milk xanthine oxidase,” J. Biol. Chem. 245, 4053). The oxygen anion radicals can also be generated by physical processes, for example with a superoxide generator (Inventor: Goldstein, N., Patent DE 195 12 228, 1995).

The NO-products of the present invention can be formed by chemical or enzyme generation, for example by NO synthesis.

In one embodiment, an aqueous solution of the respective drug is mixed immediately before administration with a corresponding quantity of a 10⁻⁵ mole/l H₂O₂ solution. The mixture is subsequently administered in form of a nasal spray or in another form. The volume of a single dose of the nasal spray is between 50 and 500 μl of a solution or a mixture of the drug and oxygen radicals as vasoactive substances.

The volume of a single dose of the nasal spray is preferably 100-200 μl. The concentration of the hydrogen peroxide in this dose is between 10⁻¹² mole/l and 10⁻¹ mole/l, preferably 10⁻⁸ mole/1 to 10⁻² mole/l, and most preferably 10⁻⁵ mole/l. The absolute quantity of the drug in the single dose is 0.0001 to 100 mg, preferably 0.1-10 mg.

In another embodiment, a mixture of xanthine oxidase and xanthine is used as a source for the oxygen anion radicals. A solution of xanthine oxidase and xanthine having a corresponding activity or concentration is mixed directly before administration with a solution of the respective drug. The volume of a single dose of the nasal spray is between 50 and 500 μl of a solution or a mixture of drug and xanthine/xanthine oxidase.

Preferably, the volume of a single dose of the nasal spray is 200 μl. The concentration of xanthine oxidase is here between the 0.01 mg/ml and 10 mg/ml, preferably between the 0.05 mg/ml, and most preferred between the 0.1 mg/ml and 1 mg/ml. The concentration of xanthine is between the 0.1 mg/ml and 100 mg/ml, preferably between 1 mg/ml and 50 mg/ml, and most preferred between 5 mg/ml and 25 mg/ml. The absolute quantity of the drug in a single dose is 0.0001 mg to 100 mg, preferably 10 mg.

Due to the large number of individual parameters in the treatment of diseases of the central nervous system (individual pharmacokinetics of the drug, cause for the disorder, etc.), exact information about the typical dosage of the corresponding drug can preferably be made based on comparative values obtained on a single individual.

The novel effect, which forms the basis for the present invention, is inter alia a synergistic therapeutic effect between vasoactive substances, for example oxygen anion radicals and/or their reaction or breakdown products, and the administered drug in a common intranasal administration, so that the dose for achieving a defined effect can be reduced by at least 50%.

To achieve the synergistic effect according to the invention, for example, the drugs promedol, metamizol, phenobarbital, dermorphin, dopamine, methadone, tramadol, Viagra™, or clonidin can be used.

The following examples demonstrate the novel enhanced efficacy of the combination of these vasoactive substances, for example oxygen anion radicals and nitrogen radicals with corresponding medication according to the present invention. Regulatory and therapeutic effects on the corresponding functions of healthy and sick organisms of animals and/or humans were investigated.

### EXAMPLE 1

The following experiments with rats show that with an endonasal administration of the composition of H₂O₂ in a concentration of 10⁻⁵ mole/i (3.4*10⁻⁴ mg/kg bodyweight) and glucose in a dose of 20 mg (i.e., 100 mg/kg bodyweight), the test animals showed a significantly reduced motivation for food. The experiments where carried out with white male rats. The animals of the control group 1 (n=13) received distilled water, the animals of the control group 2 (n=7) received a glucose solution, and the animals of the experimental group (n=9) received the composition H₂O₂+glucose. The duration of the observation was 17 days. During the first 11 days, the animals where controllably partially deprived of food (20 g dry mixed feed per animal). During the first week of the experiment, the initial motivation level for food was evaluated in the absence of the investigated preparation. The following parameters were investigated:

Latent period (LP) between the approach to the food and presentation of the food in seconds;

Time for consuming the food, in seconds (Tfut.);

Number of interruptions during food uptake (Ubr.)

After the second week, without changing the feed conditions, the preparation was introduced twice daily in a dose of 20 μl per animal, and all parameters where recorded. After the second week, the animals where fed ad libidum. No changes were made in the administration of the preparation and recordation of all parameters. The results of the investigation are listed in Tables 1 in 2. In these and other Tables, the results are shown as the average value±standard deviation.

TABLE 1
Parameters of the food motivation in the control group 1 and control group 2 in
comparison to the experimental group before and after administration of the preparation.

| Animal | Before administration of preparation | | | After administration of preparation | | |
|---|---|---|---|---|---|---|
| groups | LP [s] | T_{fut.} [s] | Ubr. [s] | LP [s] | T_{fut.} [s] | Ubr. [s] |
| Control group 1 | 76.9 ± 15.5 | 419.0 ± 25.2 | 3.8 ± 0.4 | 42.2 ± 3.7 | 489.4 ± 3.7 | 3.9 ± 0.5 |
| Intact animals | | | | | | |
| Control group 2 | 78.1 ± 14.3 | 421.6 ± 27.2 | 3.6 ± 0.5 | 48.1 ± 12.1 | 495.7 ± 11.4 | 4.1 ± 0.4 |
| Glucose | | | | | | |
| endonasal | | | | | | |
| Experiment | 83.3 ± 13.9 | 417.2 ± 16.0 | 3.8 ± 0.4 | 73.1 ± 11.9**) | 443.0 ± 17.8*) | 3.7 ± 0.6 |
| H₂O₂ + Glucose | | | | | | |
| endonasal | | | | | | |

*)= p < 0.05; and
**)= p < 0.001 as compared to control groups 1 and 2

TABLE 2
Changes in parameters of food motivation in the groups control group 1,
control group 2, and experiment. Test results before administration of the
preparation minus test results after administration of the preparation.

| Animal groups | LP | T_{fut.} | Ubr. |
|---|---|---|---|
| Control group 1 | 34.8 ± 16.0 | −70.4 ± 27.2 | −0.2 ± 0.8 |
| Intact animals | | | |
| Control group 2 | 30.0 ± 14.9 | −74.1 ± 31.6 | −0.5 ± 0.4 |
| Glucose endonasal | | | |
| Experiment | 10.2 ± 18.1 *) | −25.8 ± 21.5 | 0.1 ± 0.7 |
| H₂O₂ + Glucose | | | |
| endonasal | | | |

*) Significance p < 0.05 as compared to control groups 1 and 2

The results show that administration of the composition H₂O₂+glucose for four days (twice daily) led to a significant prolongation of the latent period before approaching the food.

### EXAMPLE 2

The experiments where conducted as in Example 1. The animals of the control group 1 (n=13) received distilled water, the animals of the control group 2 (n=6) received a glutamic acid solution, and the animals of the experimental group (n=9) received the composition H₂O₂+glutamic acid (3.4*10⁻⁴ mg/kg bodyweight) and glutamic acid 10⁻³ mole/l, i.e., 1.74*10⁻² mg/kg bodyweight). The results are listed in Tables 3 and 4.

TABLE 3
Parameters of the food motivation in the groups control group 1, control group
II and experiment before and after administration of the composition H₂O₂ + glutamic acid.

| Animal | Before administration of preparation | | | After administration of preparation | | |
|---|---|---|---|---|---|---|
| groups | LP [s] | T_{fut.} [s] | Ubr. [s] | LP [s] | T_{fut.} [s] | Ubr. [s] |
| Control group 1 | 76.9 ± 15.5 | 419.0 ± 25.2 | 3.8 ± 0.4 | 42.2 ± 3.7 | 489.4 ± 3.7 | 3.9 ± 0.5 |
| Intact animals | | | | | | |
| Control group 2 | 69.9 ± 15.9 | 414.0 ± 30.6 | 4.1 ± 0.6 | 36.3 ± 4.1 | 491.2 ± 9.4 | 3.7 ± 0.5 |
| glutamic acid | | | | | | |
| endonasal | | | | | | |
| Experiment | 45.6 ± 5.2 | 439.9 ± 5.2 | 4.8 ± 0.7 | 54.6 ± 10.0*) | 413.2 ± 22.2**) | 5.1 ± 0.6 |
| H₂O₂ + glutamic | | | | | | |
| acid | | | | | | |
| endonasal | | | | | | |

*)Significance p < 0.05 and
**)p < 0.01 as compared to control groups 1 and 2

TABLE 4
Changes in parameters of the food motivation in the groups
control group 1, control group II and experiment. Test before
administration of the administration minus test results after
administration of the preparation.

| Animal groups | LP [s] | T_{fut.} [s] | Ubr. [s] |
|---|---|---|---|
| Control group 1 | 34.8 ± 16.0 | −70.4 ± 27.2 | −0.2 ± 0.8 |
| Intact animals | | | |
| Control group 2 | 33.3 ± 13.1 | −77.2 ± 26.6 | 0.4 ± 0.6 |
| glutamic acid endonasal | | | |
| Experiment | −9.0 ± 12.4 *) | 26.7 ± 17.2 **) | −0.3 ± 0.7 |
| H₂O₂ + glutamic acid | | | |
| endonasal | | | |

*) Significance p < 0.05 and **) p < 0.01 as compared to control group

The results have shown that administration of the composition H₂O₂+glutamic acid resulted in a significant lengthening of the latent period for approaching the food and a decrease in the feeding time. It is also evident that administration of oxygen radicals in the form of H₂O₂ in a concentration of 10⁻⁵ mole/l and glutamic acid (10⁻³ mole/l) had an inhibiting effect on the food motivation in the test animals.

### EXAMPLE 3

A placebo-controlled investigation was carried out on 6 volunteers. H₂O₂+glucose mixtures in concentrations of 6.8*10⁻⁷ mg H₂O₂ and 0.01 g glucose were administered endonasally 2-3 times daily.

The investigation was carried out on healthy male and female volunteers with an increased body mass. The average age of the test persons was 49.4±8.1 years for females and 52.2±5.8 years for males. The results for reducing the bodyweight are listed in Table 5.

TABLE 5
Dynamics of reducing the bodyweight in test persons (the mixed group, 4
females) as a result of endonasal administration of the composition
H₂O₂ + glucose with 6.8 * 10⁻⁷ mg
H₂O₂ and 0.01 g glucose.

| | | | | Max. |
|---|---|---|---|---|
| | | | | reduction in |
| | Bodyweight | Bodyweight | Bodyweight | bodyweight |
| | [kg] | [kg] | [kg] | (in % of |
| Groups (Males + females) | (Initial value) | (after 14 days) | (after 56 days) | initial value) |
| Administration of | 89.6 ± 13.4 | 90.5 ± 14.3 | 90.1 ± 13.2 | — |
| placebo (n = 6) | | | | |
| Administration of | 91.8 ± 12.2 | 87.7 ± 12.9 | 78.5 ± 10.2 *)#) | 15.5% |
| the composition | | | | |
| H₂O₂ + glucose | | | | |
| (n = 6) | | | | |

*) Significance p < 0.05 as compared to placebo;
#) Significance p < 0.05 as compared to initial body mass in the respective group

The results show that the administration of the H₂O₂/glucose mixture in comparison to the placebo results in a suppression of the appetite and a consistent reduction in the bodyweight.

### EXAMPLE 4

The anti-nociceptive and/or analgesic effect of promedol (trimeperidine) was investigated as a result of the endonasal administration of the combination H₂O₂+promedol. The experiments were conducted with (raceless) sexually mature white male rats. In a pain test, the value of the critical pressure (WKD) on the rear paw of the rats was measured. The controlled pressure on the paw was generated with an analgesimeter from the company Ugo Basile in form of the Randall-Selitto test.

Comparative results in relation to the composition of the invention include, on one hand, the intraperitoneal administration of promedol without the addition of oxygen anion radicals and, on the other hand, the intraperitoneal administration of promedol with addition of oxygen anion radicals by, on one hand, inhalation with the inhalation device described in DE 41 12 459 A1 and, on the other hand, by separate endonasal administration of a liquid hydrogen peroxide solution with a concentration of 10⁻⁵ mole/l.

The administration according to the invention consisted of a mixture of xanthine-oxidase/xanthine and promedol in doses of 5, 2, 1, and 0.1 mg/kg bodyweight of the animals. The results are listed in Table 6:

TABLE 6
Enhancement of the analgesic effect of promedol in a combination with oxygen
radicals, here: Products of the xanthine-oxidase/xanthine reaction, as compared
to intraperitoneal introduction of the analgesic without NSAS (groups II-IV) and
to endonasal administration of NSAS (groups V-VII).

| | Initial | Time after injection or administration of | | | |
|---|---|---|---|---|---|
| | value of | promedol, in minutes | | | |
| Animal groups | nociception | 30 | 90 | 150 | 210 |
| I. Control group (n = 10) | 7.2 ± 1.5 | 6.2 ± 1.2 | 5.7 ± 1.3 | 5.4 ± 1.0 | 5.4 ± 1.1 |
| II. Promedol, 1.0 mg/kg; | 6.7 ± 0.9 | 6.2 ± 1.9 | 5.4 ± 1.4 | 5.0 ± 1.2 | 4.8 ± 0.9 |
| i/p. (n = 10) | | | | | |
| III. Promedol, 2.0 mg/kg; | 7.8 ± 1.6 | 10.1 ± 1.1*) | 9.3 ± 1.0*) | 8.0 ± 1.1 | 7.5 ± 0.9 |
| i/p. (n = 12) | | | | | |
| IV. Promedol, 5.0 mg/kg; | 6.4 ± 0.7 | 11.0 ± 1.0**) | 9.8 ± 1.3**) | 8.3 ± 1.7*) | 6.7 ± 1.0 |
| i/p. (n = 10) | | | | | |
| V. Promedol, 1.0 mg/kg; | 6.9 ± 1.6 | 8.1 ± 3.6*) | 8.1 ± 2.3**) | 9.6 ± 5.0**) | 5.8 ± 2.3 |
| i/p. + O₂ - inhalation | | | | | |
| (n = 10) | | | | | |
| VI. Promedol, 1.0 mg/kg; | 7.1 ± 1.8 | 8.4 ± 2.8*) | 8.7 ± 2.0**) | 8.6 ± 2.7**) | 7.3 ± 2.2 |
| i/p. + endonasal | | | | | |
| H₂O₂-administration | | | | | |
| (n = 10) | | | | | |
| VII. Combination | 6.8 ± 1.6 | 8.7 ± 3.2**) | 9.2 ± 3.0**) | 9.0 ± 2.9**) | 8.8 ± 2.2**) |
| promedol (0.1 mg/kg) + | | | | | |
| xanthine- | | | | | |
| oxidase/hypoxanthine, | | | | | |
| endonasal^{#)} (n = 12) | | | | | |

Superoxide generation speed: 0.025 μM/min;
applied total volume of H₂O₂: 440 μl (corresponds to 4 strokes of 110 μl)
*)Significance p < 0.05 and
**)p < 0.01 as compared to the initial value in each defined group;
^{#)}xanthine-oxidase-activity 0.79 units/mg protein; concentration of the hypoxanthine 0.1 mM.

The results (group VII) listed in Table 6 show a more pronounced increase of the analgesic efficacy of the analgesic in a composition with oxygen anion radicals in the endonasal administration as compared to the efficacy of promedol with parenteral introduction, as well as with the separate effect of oxygen anion radicals (endonasal) and of the analgesic (intraperitoneal). An increased efficacy of the endonasal form manifests itself in a prolongation and enhancement of the analgesic effect with a 10-50 times lower dose of the promedol as compared to other administration methods. The present experiments clearly show that oxygen anion radicals in combination with promedol increase or supra-additively enhance the potency of the analgesic effect of the analgesic.

### EXAMPLE 5

The enhancement of the therapeutic efficacy of the analgesic metamizol was investigated. A total of 5 volunteers participated in the observations. The preparation for endonasal administration consisted of a combination of hydrogen peroxide (10⁻⁶ mole/l, corresponding to a dose of 6.8*10⁻⁷ mg) and metamizol (dose of 10 mg). The results are summarized in Table 7. With all patients, the observations were made after six hours following the last administration of the pain therapy.

TABLE 7
Results with one-time endonasal administration of H₂O₂ + metamizol in 5
patients with persistent or severe pain. Dose of metamizol 10 mg.

| | | | | Pain level | | Duration |
|---|---|---|---|---|---|---|
| Patient | | Duration of | | (subjective, scale 0-5) | | of the |
| (Name | | the illness, | | Before | After | effect, |
| and age) | Clinical diagnosis | weeks | | administration | administration | hours |
| 1. male, | Posttraumatic | 7 | | 4 | 1 | 14 |
| K-r, age | headache | | | | | |
| 66 | | | | | | |
| 2. female | Post herpetic | 78 | | 4 | 1 | 22 |
| U-va, age | facial pain | | | | | |
| 58 | | | | | | |
| 3. male, | Pain after hand | 1 | day | 5 | 1 | 6 |
| R-v, age | burns | | | | | |
| 39 | | | | | | |
| 4. female | Post herpetic | 48 | | 4 | 0 | 17 |
| P-ko, age | facial pain | | | | | |
| 69 | | | | | | |
| 5. male, | Posttraumatic | 104 | | 3 | 3 | 0 |
| B-s, age | headache | | | | | |
| 32 | The basic nerve | | | | | |
| | connections | | | | | |
| | between receptors | | | | | |
| | of the nasal cavity | | | | | |
| | and brain | | | | | |
| | structures were | | | | | |
| | injured or | | | | | |
| | damaged | | | | | |

The observations 1-4 confirm the efficacy of the methods in humans. Observation 5 (no effect) confirms the important role of the nasal cavity receptors in the efficacy of endonasal administration of drugs. This is the more surprising as the non-narcotic analgesic metamizol (Dipyron) is therapeutically not effective in a nasal administration. In all patients, the administration of metamizol was not effective, neither per os in a dose of 500 mg nor endonasally in a dose of 10 mg without the addition of oxygen anion radicals. However, metamizol develops a very pronounced analgesic effect in combination with oxygen anion radicals.

### EXAMPLE 6

The two following examples (Table 8) show the efficacy of endonasal administration of the neurotransmitter dopamine in combination with different vasoactive substances: L-arginine (as a biochemical source for the nitrogen mono oxide NO), as well as hydrogen peroxide. It should be emphasized that dopamine is not capable of penetrating the blood-brain barrier when administered conventionally.

TABLE 8
Regeneration of damaged spontaneous activity through i/p
administration of haloperidole (100 mg/kg bodyweight) in rats,
caused by the onetime endonasal administration of dopamine
(0.025 mg corresponding to 0.125 mg/kg bodyweight) in
combination with L-arginine (10⁻⁵ mole/l, corresponding
to 1.75 * 10⁻⁴ mg/kg bodyweight) or
H₂O₂ (10⁻⁶ mole/l, corresponding
to 3.4 * 10⁻⁶ mg/kg bodyweight).

| | Total spontaneous |
|---|---|
| | activity |
| | (Number of |
| | measurable |
| Animal groups | movements) |
| I. Unharmed control (n = 7) | 35 ± 8 |
| II. Haloperidol i/p (n = 9) | 3 ± 3 ^{##)} |
| III. Haloperidol i/p + Dopamine endonasal (n = 5) | 2 ± 1.7 ^{##)} |
| IV. Haloperidol i/p + H₂O₂ endonasal (n = 5) | 3 ± 1.9 ^{##)} |
| V. Haloperidol i/p + H₂O₂ + Dopamine endonasal | 38 ± 7 **⁾ |
| (n = 7) | |
| VI. Haloperidol i/p + L-arginine + dopamine | 27 ± 6 **⁾ |
| endonasal (n = 6) | |

^{##)} Significance P II, III, IV vs. I < 0.01;
**⁾ = P V, VI vs. II, III, IV < 0.01.

### EXAMPLE 7

The therapeutic effect of endonasal administration of a mixture of oxygen anion radicals and dopamine was observed on sick patients with the clinical diagnosis of advanced stage Parkinson's disease (stages 2.0-3.0 according to Hoehn & Yahr). A total of 3 volunteers participated in the observations. The preparation for the endonasal administration consisted of a combination of hydrogen peroxide (10⁻⁷ mole/l) with dopamine (1 mg). All patients underwent regular standard therapy for the disease until the day of the clinical observation. The results of these observations are summarized in Table 9.

TABLE 9
Results of the observations of the therapeutic effect with one-time
administration of the preparation H₂O₂ + dopamine in patients with
Parkinson's disease (change in the motor functions and expression).

| | Tremor | | Rigor | | Walk | | | |
|---|---|---|---|---|---|---|---|---|
| Patient and | be- | | be- | | be- | | Expression | |
| duration of | fore | after | fore | after | fore | after | before | after |
| illness | treatment | | treatment | | treatment | | treatment | |
| Male E-n, | +++ | +++ | ++ | +++ | + | +++ | ++ | ++ |
| age 8 | | | | | | | | |
| Male G-o, | + | +++ | +++ | +++ | + | +++ | ++ | ++++ |
| age 3 | | | | | | | | |
| Female, N- | +++ | +++ | ++ | +++ | + | ++ | + | +++ |
| a, age 6 | | | | | | | | |

The characters +, ++, +++, and ++++ indicate a very poor, poor, good, and excellent function.

DE-PS 197 08 643 describes a moderate therapeutic effect of endonasal administration of superoxide or hydrogen peroxide in patients with tremors (Parkinson's disease). It was subsequently noted that the healing effect of oxygen anion radicals is observed predominantly during the initial stage of the disease (stages 1.0-1.5 according to Hoehn & Yahr). It was also noted in DE 197 08 643 that the healing effect of the NSAS develops not before 10 to 20 days of treatment. With the administration according to this invention, however, all patients experienced a positive healing effect within several minutes after administration, which can still be detected both subjectively and objectively (by a physician) within 6 to 72 hours.

### EXAMPLE 8

Female patient: age 61 years; clinical diagnosis: adynamic depression with mood swings. The basic clinical symptoms: apathy, remorse, concern, hostile behavior towards family members. Previous treatment: antidepressants amitriptiline, desipramine, maprotiline, as well as kavasedon, etc. The drugs were administered irregularly due to poor compatibility.

Because the condition continued to deteriorate, a composition H₂O₂+tryptophan with concentrations of 0.1 mg tryptophan and 3.4*10⁻⁴ mg H₂O₂ was administered endonasally (as a spray). The spray was administered endonasally 2 times daily over two days, each time 200 μl in each nasal cavity.

Result: The female patient experienced subjectively an improvement in the mental conditions 4 hours after the first administration. On the second and third day, apparent positive changes where noticed subjectively and objectively (by a physician). All major clinical symptoms of the disease are practically in remission. The patient reported an improvement in her ability to work, an improvement in her mood, and a decline of negative emotions. One endonasal administration per week was made during the course of a 3-week follow-up treatment. The aforedescribed improved condition continued during the 30-day observation period, without any side effects.

### EXAMPLE 9

The enhancement of the analgesic effect of dermorphine in combination with xanthine-oxidase/xanthine as compared to conventional intraperitoneal and endonasal methods for the administration of dermorphine was investigated (Table 11). The experiments were conducted with raceless white male rats. The analgesic activity was investigated by the tail flick reflex method. The reaction was recorded one hour after administering the preparation.

TABLE 11

| | Analgesic effect |
|---|---|
| Experimental groups | (in % for control) |
| 1. Intraperitoneal administration of | 12.5 ± 4.2 |
| dermorphin | |
| (0.05 mg/kg) | |
| 2. Endonasal administration of dermorphin | 26.2 ± 6.1 *) |
| (0.05 mg/kg) | |
| 3. Endonasal administration of dermorphin | 46.9 ± 6.4 **) #) |
| (0.005 mg/kg) in combination with | |
| xanthine oxidase/xanthine | |

Application volume: 20 μl
*) Significance p 2 vs. 1 < 0.01;
**) = p 3 vs. 1 < 0.01;
#) = p 3 vs. 2 < 0.01.
Enzymatic activity of xanthine oxidase/xanthine = 0.79 I.U./mg protein;
concentration of xanthine = 0.1 mM.

The results show that the oxygen anion radical-forming mixture xanthine-oxidase/xanthine in combination with the oligopeptide 9 DAFGYPS-NH2 (dermorphine) markedly enhances the antinociceptive (analgesic) effect of the analgesic with endonasal administration.

### EXAMPLE 10

The administration of phenobarbital for the treatment of epilepsy is known. Disadvantageously, however, this treatment has undesirable side effects, for example an increase in the P-450 activity in the liver and a modification of the metabolism of different drugs, as well as nausea, dizziness and the like. Phenobarbital is typically applied in a dose of approximately 100 mg.

Investigated was the effect of phenobarbital in an endonasal administration on sexually mature white mice. Both the sedative and the soporific effect of the endonasal administration of phenobarbital without oxygen anion radicals were compared with the administration of the

TABLE 12
Enhancement of the soporific effect of phenobarbital
in combination with glucose-oxidase/glucose as compared to
pure endonasal administration of phenobarbital.

| | Sleep |
|---|---|
| Experimental groups | duration (minutes) |
| Control group phenobarbital endonasal 60 mg/ml | 248.5 ± 22.2 |
| (n = 7) | |
| Experimental group | 369.8 ± 29.4 **) |
| Glucose-oxidase/glucose + phenobarbital | |
| endonasal 5 mg/ml (n = 6) | |

**) = p < 0.1.
The enzymatic activity of glucose-oxidase = 0.66 I.U./mg;
glucose concentration = 0.15 mM.

### EXAMPLE 11

The prevention of an epileptic seizure in its initial stage was investigated in a 19-year-old patient with the clinical diagnosis essential epilepsy. The symptoms, which preceded the attack, were: increased excitability, symptoms of tonic tension of the muscles, unintentional urination. The listed symptoms were always followed by an extensive epileptic seizure in this patient.

The patient was given twice within 3 minutes endonasal administrations composed of H₂O₂+phenobarbital. The effective doses of phenobarbital were always 10 mg.

Result: 3 minutes after the second administration of the composition, the symptoms of tonic tension of the muscles lessened, and the desire to urinate stopped. A moderate sleepiness developed. No epileptic seizure occurred during the following three days.

### EXAMPLE 12

It is known that the commonly used drugs, namely ergotamine, methylsergide, tricyclic antidepressants, karbamazepine, sumatriptane, etc., cause various side effects in migraine sufferers, for example nausea, vomiting, dizziness, tremor, sleepiness, skin reactions, etc.

In the present example, clinical tests were performed for preventing the beginning phase of migraine attacks in two female patients, age 36 and 28 years, both with the clinical diagnosis of migraine.

The characteristic symptoms preceding the attack were: aura (one patient), escalating unilateral headaches, and nausea. The listed symptoms in these female patients always developed into a migraine attack.

The composition H₂O₂+phenobarbital was administered endonasally in these patients. The effective dose of phenobarbital in each case was 10 mg.

Result: Approximately 5 minutes after administration, most patients reported a lessening of the listed symptoms of migraine attacks. No further migraine attacks developed in these patients during the following 72 hours.

1. Pharmaceutical product with active ingredients affecting the central nervous system, with addition of a substance active in the nasal mucous membrane for endonasal administration, wherein a combined composition of free radical products with biologically active substances are administered for potentiating the efficacy, wherein the increased efficacy occurs in combination with oxygen anion radicals (SAR) and/or nitrogen oxide active products.

2. Pharmaceutical product according to claim 1, wherein the potentiation is attained by drug-like substances and different types of metabolites, and such substances of a chemical nature.

3. Pharmaceutical product according to claim 1, wherein the potentiation is attained in conjunction with different types of free radicals (SAR, NO-radicals) and/or corresponding radical formers.

4. Pharmaceutical product according to claim 1, wherein the substances active in the nasal mucous membrane are perhydroxyl radicals, hydrogen peroxide, hydroperoxide radicals or their hydrate clusters.

5. Pharmaceutical product according to claim 1, wherein the substances active in the nasal mucous membrane are forms of nitrogen monoxide (NO) and their precursors or reaction products.

6. Pharmaceutical product according to claim 1, wherein the substances active in the nasal mucous membrane are biochemical, physiological vaso-dilators, preferably arginin, bradykinin, urea or eicosatric acid-derivates.

7. Pharmaceutical product according to claims 1, wherein utilizing a mixture comprising substances active in the nasal mucous membrane in a concentration of 10⁻¹² mole/l to 10⁻¹ mole/l.

8. Pharmaceutical product according to claims 1, wherein utilizing a mixture comprising substances active in the nasal mucous membrane in a concentration of 10⁻⁵.

9. Pharmaceutical product according to claims 1 wherein conventional drug substances are included in a dose of 0.001 mg to 100 mg per dosage unit.

10. Pharmaceutical product according to claims 1, wherein the metabolite is included in a dose of 0.0001 mg to 100 mg per dosage unit.

11. Pharmaceutical product according to claims 1, wherein the drug substances are promedol, metamizol, phenobarbital, methadone, tramadol, ASS or sildenafil.

12. Pharmaceutical product according to claim 1, wherein the metabolite is tryptophan, gamma-amino butyric acid, oxytocin, dermorphin, cyclic GMP, glucose, dopamine, or L-dopa.

13. Pharmaceutical product according to claims 1, wherein one or more of its active components are present in the composition as liposomes and/or nanosomes.

14. Pharmaceutical product according to claims 1 wherein one or more of its active components are present in the composition in a form different from the solution.

15. Pharmaceutical product according to claims 1, wherein pharmaceutically acceptable, auxiliary substances are present in the composition.

16. Pharmaceutical product according to claims 1, wherein the auxiliary substances are stabilizers, antioxidants, pH regulators, osmo-regulators or antimicrobial substances, which are present in the product in combination with a pharmaceutical substance adequate for its administration.

17. Pharmaceutical product according to claims 1, wherein the product is a spray that can be endonasally administered.
